(19) United States
(12) Patent Application Publication
(10) Pub. No.: US 2010/0239559 A1
(43) Pub. Date: Sep. 23, 2010

(54) Novel nutritional food products for improved digestion and intestinal absorption

(75) Inventors: Steven Freedman, Chestnut Hill, MA (US); Deborah A. DaSilva, Sount Easton, MA (US)

Correspondence Address: Ralph A. Loren;Edwards Angell Palmer & Dodge LLP, P.O. Box 55874, Boston, MA, 02205, US

(73) Assignee: Beth Israel Deaconess Medical Center, Inc., Boston, MA (US)

(21) Appl. No.: 12/587,579

(22) Filed: Oct. 9, 2009

Related U.S. Application Data
(63) Continuation of application No. PCT/US08/04351, filed on Apr. 3, 2008.
(60) Provisional application No. 60/923,503, filed on Apr. 13, 2007.

(51) Int. Cl. A61K 38/46; A61K 38/47; A61K 38/48; C12N 9/20; C12N 9/26; C12N 9/48; A61P 1/14

(52) U.S. Cl. 424/94.61; 424/94.6; 424/94.63; 435/198; 435/201; 435/212

(57) ABSTRACT

The present invention is directed to novel food products, e.g., nutritional food products and infant formula, which contain one ore more enzymes selected from lipase, protease, and amylase that have been formulated/stabilized to have sustained stability in an aqueous medium. Such formulations are intended to provide a greater degree of compliance based on their ability to be incorporated into aqueous media while avoiding unstable breakdown of the enzyme and large overdosing due to expected breakdown when exposed to an aqueous environment, including saliva. Further described in the invention are additives packaged with instructions for combination with an aqueous medium, and instructions for the administration of the resulting mixture to a subject. In certain embodiments, enzyme insufficient patients, e.g., infants and elderly persons, would find particular benefit from the food products described herein.

### BACKGROUND OF THE INVENTION

Digestive health is considered to be one of the most critical factors in the proper absorption of fats, proteins, carbohydrates, and vitamins from ingested foods necessary for proper body functions. To this end, supplements have been developed and used, not only to augment the body's need for full and proper nutrition, but also to assist the body in utilizing nutrients found in consumed food. For example, known digestive supplements may include one or more of the following components: soluble and insoluble fibers, herbal concentrates, beneficial microflora (probiotics, e.g., acidophilus, such as lactobacillus acidophilus), fruits or products derived from fruits (e.g., apple and papaya, including bran and pectin), and psyllium seed (Indian husks).

Additionally, and often more successful, enzyme supplements/additives have also been used to assist in overall digestion and digestive health, and include: alpha-galactosidase, amylase, cellulase, glucoamylase, invertase, lactase, lipase, malt diastase (aka maltose), protease (e.g., protease blends including one or more of alkaline, neutral and acid proteases, and peptidase), beta-glucanase, pectinase, phytase, and xylanase. However, while certain enzymes have proven to be effective in assisting in digestion, compliance using existing formulations, which typically include bulky “horse” pills or awkwardly tasting and difficult to swallow dry powders, continues to be problematic.

Alternatively, with respect to certain digestive disorders where an insufficiency in a particular enzyme may be effectively treated by a supplement, e.g., lactose intolerance, the issue of administration has seen resolution with the advent of different means of administration and/or formulation: from sweet tasting chewable pills to the pre-activation of the food product to allow for molecular breakdown, e.g., of lactose, prior to ingestion. Unfortunately, however, for other enzymes, such as lipase, such resolutions have not been successful due to the significant instability of the enzyme. In fact, it was only recently that Margolin et al. (U.S. Pat. No. 6,541,606) described limited shelf stable formulations that contained enzyme crystals, such as lipase, with or without an excipient.

Accordingly, even with the advent of new dietary supplements that have sought to improve or enhance the ability of a person to digest or absorb nutrients from consumed food, a need still exists for more convenient forms of enzyme supplements that are stable and would provide improved compliance.

### SUMMARY OF THE INVENTION

As such, the present invention is directed to novel food products, e.g., nutritional food products and infant formula, which contain one ore more enzymes selected from lipase, protease, and amylase that have been formulated/stabilized to have sustained stability in an aqueous medium. Such formulations are intended to provide a greater degree of compliance based on their ability to be incorporated into aqueous media while avoiding unstable breakdown of the enzyme and large overdosing due to expected breakdown when exposed to an aqueous environment (including saliva). Further described in the invention are packaged additives, packaged with instructions for combination with an aqueous medium, and instructions for the administration of the resulting mixture to a subject. In certain embodiments, enzyme insufficient subjects, e.g., infants and elderly persons, would find particular benefit from the food products described herein.

Accordingly, the invention relates to a nutritional product composition comprising an enzyme selected from the group consisting of a lipase, an amylase, a protease, and any combination thereof, which has been formulated for sustained stability in an aqueous medium, e.g., an infant formula or a nutritional drink product. The nutritional product composition also comprises a nutritional supplement. Moreover, in particular embodiments of the invention, the composition may be formulated for administration to an infant or an elderly person.

The invention is also directed to an infant formula composition. The infant formula composition comprises infant formula and an enzyme selected from the group consisting of a lipase, an amylase, a protease, and any combination thereof, which is formulated for sustained stability in the infant formula.

In another aspect, the invention relates to a packaged additive comprising an enzyme selected from the group consisting of a lipase, an amylase, a protease, and any combination thereof, formulated for sustained stability in an aqueous medium, as well as instructions for mixing the additive with the aqueous medium and administration of the resulting product mixture to a subject.

The invention also pertains to a packaged infant formula additive. This package comprises an enzyme selected from the group consisting of a lipase, an amylase, a protease, and any combination thereof, formulated for sustained stability in infant formula, as well as instructions for mixing the additive with infant formula and administration of the resulting product mixture to an infant.

Additionally, the invention relates to a composition useful for increased intestinal absorption of a nutrient. The composition comprises a low-dose quantity of an enzyme selected from the group consisting of a lipase, an amylase, a protease, and any combination thereof, which is formulated for low-dose administration of the enzyme to a subject (e.g., an infant) in aqueous medium.

The invention also features a digestion enhancement composition. The composition comprises a low-dose quantity of an enzyme selected from the group consisting of a lipase, an amylase, a protease, and any combination thereof, and is formulated for low-dose administration of the enzyme to a subject (i.e., an infant) in aqueous medium.

Another aspect of the invention is directed to a method of increasing intestinal absorption of a nutrient. The method comprises administering to an infant an enzyme selected from the group consisting of a lipase, an amylase, a protease, and any combination thereof, which is formulated for sustained stability in an aqueous medium. Moreover, the enzyme that is administered is also adapted for administration to an infant in the aqueous medium (e.g., infant formula), such that the intestinal absorption of the nutrient in the infant is increased.

The invention also pertains to a method of increasing intestinal absorption of a nutrient. The method comprises administering to a subject a low-dose quantity of an enzyme selected from the group consisting of a lipase, an amylase, a protease, and any combination thereof, which is formulated for low-dose administration of the enzyme in an aqueous medium. Furthermore, the administered enzyme is also adapted for administration to a subject (i.e., an infant) in an aqueous medium (e.g., infant formula), such that the intestinal absorption of the nutrient in the subject is increased.

The invention features to a method of increasing food digestion. The method comprises administering to an infant an enzyme selected from the group consisting of a lipase, an amylase, a protease, and any combination thereof, where the enzyme is adapted for administration to an infant in an aqueous medium (e.g., infant formula), such that the digestion of food ingested by the infant is increased.

An additional aspect of the invention pertains to a method of increasing food digestion. The method comprises administering to a subject a low-dose quantity of an enzyme selected from the group consisting of a lipase, an amylase, a protease, and any combination thereof, which is adapted for administration to a subject (i.e., an infant) in an aqueous medium (e.g., infant formula). Moreover, the enzyme is also formulated for low-dose administration of the enzyme, such that the digestion of food ingested by the subject is increased.

### DETAILED DESCRIPTION OF THE INVENTION

The present invention relates to compositions containing at least one lipase, amylase, protease, or combination thereof, formulated to have sustained stability in aqueous medium, as well as uses therefor. Furthermore, the use of these compositions as food products, e.g., nutritional product compositions and infant formula, for supplementing the diet of an individual (e.g., in need thereof) is described herein to be beneficial for increasing digestion and intestinal absorption of nutrients from ingested food. For example, the supplementation of infant formula with enzymes, such as pancreatic enzymes, may be of benefit to infants, especially within the first several months of life.

Enzymes including lipase, protease, and amylase are well known for their roles in digestion (i.e., fatty acid breakdown, protein breakdown, and carbohydrate breakdown, respectively), and in turn in their roles in allowing for proper intestinal nutrient absorption. However, large populations of people suffer from general insufficiency in these digestive enzymes, and particularly lipase. Moreover, much of this population of people includes those at the very beginning of life, infants, as well as the elderly.

In particular, fats are an essential component of the human diet. And while fats may be considered compact sources of energy (providing about 50% of the caloric requirement for an infant), fats are also known to be essential constituents of neural and retinal tissues. To this end, breakdown of dietary fats (e.g., triacylglycerols; monoacylglycerols; phospholipids; and cholesterol esters) requires sufficient digestive enzymes, e.g., lipases, which operate throughout the gastrointestinal tract to break down the fats and allow proper absorption of nutrients in the intestines.

However, for example, in contrast to the standard enzyme sufficient human adult, the newborn infant's exocrine pancreas remains under-developed, and thus is unable to secrete the appropriate amount of enzymes such as lipase, even in response to amino acids that would typically stimulate the endocrine system to increase hormonal secretions. In similar fashion, many elderly humans have similar insufficiencies in their enzyme production, e.g., lipase.

Before further description of the present invention, and in order that the invention may be more readily understood, certain terms have been first defined and collected here for convenience.

### I. Definitions

The term “administering” as used herein, describes all forms of art-recognized oral administration, e.g., by mouth, by gastric feeding tube, duodenal feeding tube, or gastrostomy.

The term “biocompatible polymers” describes polymers that are non-antigenic (when not used as an adjuvant), non-carcinogenic, non-toxic and which are not otherwise inherently incompatible with living organisms. Examples include: poly (acrylic acid), poly (cyanoacrylates), poly (amino acids), poly (anhydrides), poly (depsipeptide), poly (esters) such as poly (lactic acid) or PLA, poly (lactic-co-glycolic acid) or PLGA, poly (t -hydroxybutryate), poly (caprolactone) and poly (dioxanone); poly (ethylene glycol), poly ((hydroxypropyl)methacrylamide, poly [(organo)phosphazene], poly (ortho esters), poly (vinyl alcohol), poly (vinylpyrrolidone), maleic anhydride-alkyl vinyl ether copolymers, pluronic polyols, albumin, alginate, cellulose and cellulose derivatives, collagen, fibrin, gelatin, hyaluronic acid, oligosaccarides, glycaminoglycans, sulfated polysaccarides, blends and copolymers thereof.

The term “biodegradable polymers” describes polymers that degrade by hydrolysis or solubilization. Degradation can be heterogeneous, occurring primarily at the particle surface, or homogeneous, degrading evenly throughout the polymer matrix, or a combination of such processes. The term “enzyme crystal,” is intended to have its art-recognized meaning.

Accordingly, “enzyme crystal” describes protein molecules arranged in a crystal lattice. Enzyme crystals contain a pattern of specific protein--protein interactions that are repeated periodically in three dimensions (C. S. Barrett, Structure of Metals, 2nd ed., McGraw-Hill, New York, 1952, p. 1). The enzyme crystals of this invention do not include amorphous solid forms or precipitates of enzymes, such as those obtained by lyophilizing an enzyme solution. Crystals display characteristic features including a lattice structure, characteristic shapes and optical properties such as refractive index.

The term “crystal formulations” or “enzyme crystal formulations” are used interchangeably herein, and describe a combination of the enzyme crystals described as useful in this invention and one or more ingredients or excipients, such as sugars and biocompatible polymers. Examples of excipients are described in the Handbook of Pharmaceutical Excipients, published jointly by the American Pharmaceutical Association and the Pharmaceutical Society of Great Britain. Furthermore “enzyme crystal formulations” may also comprise a combination of enzyme crystals encapsulated within a polymeric carrier to form coated particles, or a combination of such encapsulated crystals with an excipient. The coated particles of the protein crystal formulation may have a spherical morphology and be microspheres of up to 500 micrometers in diameter or they may have some other morphology and be microparticulates.

For the purposes of this application, “ enzyme crystal formulations” are intended to be distinct from the compositions, i.e., food products, of the invention, in that the enzyme crystal formulations are one component of the compositions of the invention. For example, the enzyme crystal formulations useful in the present invention may be combined with at least a second ingredient, e.g., a nutritional supplement or infant formula, to make the compositions of the invention.

The macromolecule crystals have the longest dimension between about 0.01 μm and about 500 μm, alternatively between about 0.1 μm and about 100 μm. The most preferred embodiment is that the enzyme crystal or enzyme crystal formulation components are between about 50 μm and about 100 μm in their longest dimension. Such crystals may have a shape selected from the group consisting of: spheres, needles, rods, plates, such as hexagons and squares, rhomboids, cubes, bipyramids and prisms.

The term “infant” has its common meaning and specifically includes infants from greater than 14 weeks preterm up to about 2 years old.

The term “infant formula” is art-recognized and describes the modern artificial substitute for human breast milk, designed for infant consumption, and usually based on either cow milk or soy milk. Moreover, the medical community considers infant formula nutritionally acceptable for infants under the age of one year. According to

Wikipedia (http://en.wikipedia.org), most of the world's supply of infant formula is produced in the United States. The nutrient content is regulated by the American Food and Drug Administration (FDA) based on recommendations by the American Academy of Pediatrics Committee on Nutrition, and must be include: protein; fat; linoleic acid; vitamins: A, C, D, E, K, thiamin (B1), riboflavin (B2), B6, B12; niacin; folic acid; pantothenic acid; calcium; metals: magnesium, iron, zinc, manganese, copper; phosphorus; iodine; sodium chloride; and potassium chloride (formulas not made with cow's milk must include biotin, choline, inositol). Additionally, infant formula intended for use in the nutritional diet of premature infants, e.g., products that are commercially available for premature infants (which may or may not have increased nutrient content than formula useful for mature infants), is within the scope of the term “infant formula.”

The term “liquid polymer” describes a pure liquid phase synthetic polymer, such as polyethylene glycol (PEG), in the absence of aqueous or organic solvents.

The term “low-dose” as used in the expression “low-dose quantity” or “low dose administration” is used herein to describe the quantity of the dose needed to effect a specific change, and therefore the size of the dose administered in a composition of the present invention. Moreover, the size of the dose included within the compositions of the present invention may be significantly smaller than currently administered to treat the same disorder or alleviate the same amount and type of symptoms due to the enhanced stability in aqueous media, including saliva. For example, the magnitude of the dosage of the enzyme described herein may be 10 times less than existing treatment using the same enzyme, e.g., 9 times less, e.g., 8 times less, e.g., 7 times less, e.g., 6 times less, e.g., 5 times less, e.g., 4 times less, e.g., 3 times less, e.g., 2 times less, e.g., 1.75 times less, e.g., 1.5 times less, e.g., 1.25 times less.

Low-dose quantities of the enzymes of the present invention may be less than 10,000 units of enzyme, e.g., less than 9,000 units of enzyme, e.g., less than 9,000 units of enzyme, e.g., less than 9,000 units of enzyme, e.g., less than 9,000 units of enzyme, e.g., less than 9,000 units of enzyme, e.g., less than 9,000 units of enzyme, e.g., less than 9,000 units of enzyme, e.g., less than 9,000 units of enzyme, e.g., less than 9,000 units of enzyme, e.g., less than 9,000 units of enzyme,

The term “nutritional supplement” is art-recognized, and may include supplements that are intended to supply nutrients (such as, vitamins, minerals, fats, carbohydrates, proteins , or even water) that are missing or not consumed in sufficient quantity in a subject's diet. In certain embodiments, the nutient is an essential nutrient, which is required for normal body functioning, and which cannot be synthesized by the body.

In addition, the nutritional supplements described herein include therapeutic foods, i.e.,. food designed for specific, usually nutritional, therapeutic purposes (such as Ensure, a fortified milkshake drink designed primarily for the elderly; Fortisip, a milkshake-style drink similar to Ensure; and Plumpy'nut, a peanut based food designed for emergency feeding of severely malnourished children). Nutritional supplements that may be in liquid form, such as Ensure, may also be capsulated by the language “nutritional drink product.”

In certain embodiments, the nutritional supplements described herein may also be in the form of a combination product that includes existing therapies, wherein the food composition of the present invention would be considered a combination therapy. For example, the nutritional supplement may be soluble and insoluble fibers, herbal concentrates, beneficial microflora (probiotics, e.g., acidophilus, such as lactobacillus acidophilus), fruits or products derived from fruits (e.g., apple and papaya, including bran and pectin), and psyllium seed (Indian husks); as well as enzymes selected from alpha-galactosidase, amylase, cellulase, glucoamylase, invertase, lactase, lipase, malt diastase (aka maltose), protease (e.g., protease blends including one or more of alkaline, neutral and acid proteases, and peptidase), beta-glucanase, pectinase, phytase, and xylanase.

The term “polymer” is intended to have its art-recognized meaning. Accordingly, the term “polymer” describes a large molecule built up by the repetition of small, simple chemical units. The repeating units may be linear or branched to form interconnected networks. The repeat unit is usually equivalent or nearly equivalent to the monomer.

The tem “polymeric carriers,” as used herein, describes polymers used for encapsulation of protein crystals for delivery of proteins, including biological delivery. Such polymers include biocompatible and biodegradable polymers. The polymeric carrier may be a single polymer type or it may be composed of a mixture of polymer types. Polymers useful as the polymeric carrier, include for example, poly (acrylic acid), poly (cyanoacrylates), poly (amino acids), poly (anhydrides), poly (depsipeptide), poly (esters) such as poly (lactic acid) or PLA, poly (lactic-co-glycolic acid) or PLGA, poly (B-hydroxybutryate), poly (caprolactone) and poly (dioxanone); poly (ethylene glycol), poly ((hydroxypropyl)methacrylamide, poly [(organo)phosphazene], poly (ortho esters), poly (vinyl alcohol), poly (vinylpyrrolidone), maleic anhydride-alkyl vinyl ether copolymers, pluronic polyols, albumin, natural and synthetic polypeptides, alginate, cellulose and cellulose derivatives, collagen, fibrin, gelatin, hyaluronic acid, oligosaccharides, glycaminoglycans, sulfated polysaccharides, or any conventional material that will encapsulate protein crystals.

The term “shelf stability” is art-recognized, and describes nature of an enzyme, enzyme formulation, or composition of this invention with respect the amount of time the enzyme, formulation, or composition remains stable, i.e., resisting significant loss of specific activity and/or changes in secondary structure from the native protein over time incubated under specified conditions.

The term “subject” describes human and non-human animals that are capable of benefiting from the compositions and methods of the invention. The term “non-human animals” of the invention includes all vertebrates, e.g., mammals, e.g., rodents, e.g., mice, and non-mammals, such as non-human primates, sheep, dog, cow, chickens, amphibians, reptiles, etc. Preferred human animals include human patients suffering from or prone to suffering digestive disorders or insufficient absorption of nutrients. In certain embodiments, the subjects are pancreatic insufficient subjects, e.g., infant subjects or elderly subjects, having less than sufficient amount of enzyme to properly digest ingested food and/or properly absorb nutrients. In a particular embodiment, the infant subject is a premature infant. In certain embodiments, the subject is less than 2 years of age, e.g., less than 20 months of age, e.g., less than 18 months of age, e.g., less than 16 months of age, e.g., less than 14 months of age, e.g., less than 12 months of age, e.g., less than 10 months of age, e.g., less than 8 months of age, e.g., less than 6 months of age, e.g., less than 5 months of age, e.g., less than 4 months of age, e.g., less than 3 months of age, e.g., less than 2 months of age, e.g., less than 1 month of age, e.g., greater than 1 week premature, e.g., greater than 2 weeks premature, e.g., greater than 3 weeks premature, e.g., greater than 4 weeks premature, e.g., greater than 5 weeks premature, e.g., greater than 6 weeks premature, e.g., greater than 8 weeks premature, e.g., greater than 10 weeks premature, e.g., greater than 12 weeks premature, e.g., greater than 14 weeks premature. In certain embodiments, the subject is greater than 55 years of age, e.g., greater than 60 years of age, e.g., greater than 65 years of age, e.g., greater than 70 years of age, e.g., greater than 75 years of age, e.g., greater than 80 years of age.

In certain embodiments of the invention the subject, e.g., infant, is not afflicted with cystic fibrosis. In certain embodiments of the invention, the compositions are for use with non-cystic fibrosis afflicted infants.

The language “sustained stability in an aqueous medium” as used herein, describes the increased stability, e.g., decreased breakdown or inactivating event, of the enzymes (noted to be useful in the compositions of the present invention) in an aqueous medium with respect to formulations of the same enzymes that are not similarly formulated to have stability in an aqueous medium. In fact, due to enhanced stability of the enzymes described herein, the compositions of the invention may also comprise the aqueous medium. Examples of aqueous media (which is art-recognized to have some component of water) include, for example, an infant formula, a nutritional drink product, breast milk, cow's milk, or evaporated milk. In certain embodiments, the aqueous medium is a pseudo-liquid (less free flowing yet having a water base) including foods, such as applesauce and baby food. Moreover, it should be noted that the enzymes useful in the present invention need to have stability in only one selected medium, i.e., not all media. It is also important to point out that the characterization of sustained stability in an aqueous medium is independent from the form in which the food composition of the present invention is packaged or administered.

Furthermore, stability may be measured as sustained if the enzyme is insubstantially changed in aqueous medium within the time frame typically required for use of the enzyme. Insubstantially changed will be based upon the actual amount necessary to effect the change desired. Accordingly, the stability of the enzyme is considered insubstantially changed if there is less than 40% breakdown of the enzyme, e.g., less than 35% breakdown, e.g., less than 30% breakdown, e.g., less than 25% breakdown, e.g., less than 20% breakdown, e.g., less than 15% breakdown, e.g., less than 10% breakdown, e.g., less than 5% breakdown, e.g., less than 4% breakdown, e.g., less than 3% breakdown, e.g., less than 2% breakdown, e.g., less than 1% breakdown, e.g., less than 0.5% breakdown.

### II. Compositions of the Invention

The novel compositions of the present invention are directed to food products, i.e., nutritional food products and infant formula, which explicitly contain at least one lipase, protease or amylase enzyme, each characterized by its ability to be formulated to have sustained stability in an aqueous medium. As such, enzymes for inclusion in the food products of the present invention may be produced or isolated by any art-recognized means, provided that the enzyme in its formulated state has a sustained stability in an aqueous medium. It should be noted, however, that the sustained stability may result from the enzyme itself, the manner of formulation, or a combination thereof.

### A. Components of the Compositions of the Invention

The enzymes useful for incorporation into the compositions of the invention may be synthesized or obtained from any source that produces a viable enzyme, as well as naturally or synthetically modified (i.e., provided that this modification does not significantly affect the ability of the enzyme to perform it's intended function). For example, the enzymes may be isolated using bacteria, a fungus, or a plant, or may be cultured from a cell line that is derived from an animal, e.g., a mammal. In certain embodiments, the enzyme is derived from the group consisting of bacteria cultures and mammalian cultures. In particular embodiments, the enzyme is derived from Candida rugosa or functional mutants thereof. In other particular embodiments, the enzyme is derived from Pseudomonas cepacia or functional mutants thereof.

As noted herein, the enzymes incorporated into the food products of the invention include the general categories of lipases, proteases and amylases. In particular, the subcategories of these general classes of enzymes that are particularly useful in the present invention are defined by their ultimate location/utility in the digestion tract (and related organs, such as the liver, gallbladder, and pancreas), including the mouth, throat, and gastrointestinal tract. For example, the lipase enzymes that are useful in the present invention include pancreatic lipase, lysosomal lipase, gastric lipase (although both lingual and gastric lipases may exist, and hereafter they will be referred to collectively as “preduodenal” lipase), endothelial lipase, hepatic lipase, lipoprotein lipase, and a diverse array of phospholipases. In certain embodiments, the lipases are categorized as preduodenal, pancreatic, and breast milk lipases. In certain embodiments, the lipase enzymes that are not useful in the present invention include hepatic lipase, and lipoprotein lipase.

Additionally, the amylase enzyme may be selected from α-amylase, β-amylase, γ-amylase, acid α-glucosidase, and salivary amylase (ptyalin), while the protease enzyme may be selected from serine protease, threonine protease, cysteine protease, aspartic acid protease (e. g., plasmepsin), metalloprotease, and glutamic acid protease.

The enzymes may have increased performance in the presence of additional cofactors administered to a subject in addition to the enzymes described herein. Such administration is intended to be within the scope of this invention. In certain embodiments, the cofactor is added into the composition of the invention. Alternatively, the cofactor may be provided in a separate administration step in a separate composition. Accordingly, in particular embodiments, a composition of the invention may further comprise additional cofactors selected for their ability to assist in enzyme function. Such cofactors may include colipase, one or more bile salts, or certain anions (such as chlorine or bromine). In a specific embodiment, the cofactor is a bile salt.

In particular embodiments, the enzyme is a pancreatic enzyme, e.g., a pancreatic lipase enzyme. The enzyme may be present in the composition in a low-dose quantity. Moreover, this low-dose quantity is intended to reduce the burden on the subject ingesting the enzyme and increase compliance for regimens including enzyme supplementation. The composition may further comprise a second lipase that is selected from of a pre-duodenal lipase, a breast milk lipase, or a combination thereof.

In certain embodiments, compositions of the present invention contain uncross-linked enzyme crystals, cross-linked enzyme crystals, or formulations containing them, which may also have been encapsulated within a polymeric carrier to form coated particles. In fact, specific examples of enzymes that may be useful in the food product compositions of the present invention may be more completely described in U.S. Pat. No 6,541,606, which is incorporated herein by reference in its entirety. In a specific embodiment, the compositions of the present invention may comprise the known drug candidate, ALTU-135, currently in clinical trials as a treatment for pancreatic insufficiency. Noting that ALTU-135 is designed to improve fat, protein, and carbohydrate absorption in pancreatic insufficient individuals through the use of three enzymes: lipase, protease and amylase that are delivered in a consistent ratio and; and has been administered in a highly concentrated form to reduce patient burden and increase patient compliance.

The compositions of the invention may employ a stable form of an active enzyme, i.e., a crystalline form, and either (1) adding ingredients or excipients where necessary to stabilize dried crystals or (2) encapsulating the enzyme crystals or crystal formulations within a polymeric carrier to produce an enzyme composition that contains each crystal and subsequently allows the release of active protein molecules.

Moreover, the enzyme crystal(s) may be encapsulated using a variety of polymeric carriers having unique properties suitable for delivery to different and specific environments or for effecting specific functions. The rate of dissolution of the compositions and, therefore, delivery of the active enzyme can be modulated by varying crystal size, polymer composition, polymer cross-linking, crystal cross-linking, polymer thickness, polymer hydrophobicity, polymer crystallinity or polymer solubility.

In certain embodiments, the addition of ingredients or excipients to the crystals of the enzyme(s) described herein or the encapsulation of the enzyme crystals or crystal formulations results in further stabilization of the enzyme constituent. In a particular embodiment of this invention, the stability of the enzyme to be used in the food products of the invention may also derive from the preparation of the enzyme in combination with an excipient. Excipients that may be useful in the present invention include, for example, the excipients described in U.S. Pat. No. 6,541,606, which are appropriate for the administration indicated in the present invention.

The invention may include ingredients or excipients such as: salts of 1) amino acids such as glycine, arginine, aspartic acid, glutamic acid, lysine, asparagine, glutamine, proline, 2) carbohydrates, e.g. monosaccharides such as glucose, fructose, galactose, mannose, arabinose, xylose, ribose and 3) disaccharides, such as lactose, trehalose, maltose, sucrose and 4) polysaccharides, such as maltodextrins, dextrans, starch, glycogen and 5) alditols, such as mannitol, xylitol, lactitol, sorbitol 6) glucuronic acid, galacturonic acid, 7) cyclodextrins, such as methyl cyclodextrin, hydroxypropyl-β-cyclodextrin and alike 8) inorganic salts, such as sodium chloride, potassium chloride, magnesium chloride, phosphates of sodium and potassium, boric acid ammonium carbonate and ammonium phosphate, and 9) organic salts, such as acetates, citrate, ascorbate, lactate 10) emulsifying or solubilizing agents like acacia, diethanolamine, glyceryl monostearate, lecithin, monoethanolamine, oleic acid, oleyl alcohol, poloxamer, polysorbates, sodium lauryl sulfate, stearic acid, sorbitan monolaurate, sorbitan monostearate, and other sorbitan derivatives, polyoxyl derivatives, wax, polyoxyethylene derivatives, sorbitan derivatives 11) viscosity increasing reagents like, agar, alginic acid and its salts, guar gum, pectin, polyvinyl alcohol, polyethylene oxide, cellulose and its derivatives propylene carbonate, polyethylene glycol, hexylene glycol, tyloxapol. A further preferred group of excipients or ingredients includes sucrose, trehalose, lactose, sorbitol, lactitol, inositol, salts of sodium and potassium such as acetate, phosphates, citrates, borate, glycine, arginine, polyethylene oxide, polyvinyl alcohol, polyethylene glycol, hexylene glycol, methoxy polyethylene glycol, gelatin, hydroxypropyl-β-cyclodextrin. In certain embodiments, the ratio of said excipient to the enzyme formulations that may be incorporated into the compositions of invention is between 1:99 and 10:90 (W/W). In particular embodiments, the excipient is selected from the group consisting of sucrose, trehalose, lactitol, gelatin, hydroxypropyl-β-cyclodextrin, methoxypolyethylene glycol and polyethylene glycol.

Enzyme crystal formulations to be incorporated into the food product compositions according to this invention may also comprise any conventional carrier or adjuvant used in pharmaceuticals, personal care compositions or veterinary formulations that are appropriate for the administration described in the present invention. In certain embodiments, the appropriate carrier or adjuvant is generally art-recognized or listed in U.S. Pat. No. 6,541,606.

### B. Food Product Compositions of the Invention

Accordingly, one embodiment of the invention is directed to a food product composition. The composition comprises an enzyme selected from the group consisting of a lipase, an amylase, a protease, and any combination thereof, formulated for sustained stability in aqueous medium, as well as a food additive. “Food additives” are art-recognized and include all substances added to food to preserve flavor or improve its taste and appearance. Exemplary food additives include acids, acidity regulators, anticaking agents, antifoaming agents, antioxidants, bulking agents, food coloring, color retention agents, emulsifiers, flavors, flavor enhancers, flour treatment agents, humectants, preservatives, stabilizers, sweeteners, and thickeners.

Another embodiment of the invention relates to a nutritional product composition comprising an enzyme selected from the group consisting of a lipase, an amylase, a protease, and any combination thereof, which is formulated for sustained stability in an aqueous medium (e.g., an infant formula, a nutritional drink product, breast milk, cow's milk, or evaporated milk). The nutritional product composition also comprises a nutritional supplement. Moreover, the composition may be formulated for administration to an infant or an elderly person. The nutritional product may be a nutrition bar or in powder form.

In another aspect, the invention is directed to an infant formula composition. The infant formula composition comprises infant formula and an enzyme selected from the group consisting of a lipase, an amylase, a protease, and any combination thereof. In addition, the enzyme is formulated for sustained stability in the infant formula. In certain embodiments, the composition is adapted for use in infant formula selected from the group consisting of powder formula, concentrated formula, and ready to feed (use) formula. In certain embodiments, infant formula within the scope of this invention includes commercially available infant formula. For example, the infant formula of the invention may include formula manufactured by Mead Johnson, e.g., Enfamil®; manufactured by Nestlé; e.g., Good Start Supreme, Good Start 2 with DHA and ARA, Good Start 2 Soy DHA and ARA, Good Start Supreme DHA and ARA, and Good Start Essentials; manufactured by Ross Pediatrics, e.g., Similac; manufactured by Wyeth Nutrition, e.g., S-26 Gold, Promil Gold, Progress Gold, S-26, Promil, Promil Kid, Bonna, Bonamil, Bonakid 1+, Bonakid 3+, and Nursoy; manufactured by Bright Beginnings; manufactured by Gerber Products Company; and manufactured by Royal Numino Dumex, Milupa.

The infant formula may comprise components selected from the group consisting of water, enzymatically Hydrolyzed Reduced Minerals Whey Protein Concentrate (From Cow's Milk), Enzymatically Hydrolyzed Soy Protein Isolate, Nonfat Dry Milk, Corn Syrup, Vegetable Oils (Palm Olein, Soy, Coconut, High-Oleic Sunflower Oil, High-Oleic Safflower), Lactose, Sucrose, Corn Maltodextrin, and less than 1.5% of: Potassium Citrate, Potassium Phosphate, Calcium Chloride, Calcium Phosphate, Sodium Citrate, Magnesium Chloride, Ferrous Sulfate, Zinc Sulfate, Sodium Chloride, Copper Sulfate, Potassium Iodide, Manganese Sulfate, Calcium Citrate, Potassium Chloride, Sodium Citrate, Soy Lecithin, Carrageenan, Vitamins (Sodium Ascorbate, Inositol, Choline Chloride, Choline Bitartrate, Alpha-Tocopheryl Acetate, Niacinamide, Calcium Pantothenate, Riboflavin, Vitamin A Acetate, Pyridoxine Hydrochloride, Thiamine Mononitrate, Folic Acid, Phylloquinone, Biotin, Vitamin D3, Vitamin B12), Taurine, Nucleotides, e.g., naturally found in breast milk (Cytidine 5′-Monophosphate, Disodium Uridine 5′-Monophosphate, Adenosine 5′-Monophosphate, Disodium Guanosine 5′-Monophosphate), L-Carnitine, M. alpina Oil, C. cohnii Oil, Sodium Selenate, Ascorbyl Palmitate, Mixed Tocopherols, L-Methionine, and combinations thereof.

For example (and solely for the purpose of exemplification), the instant formula comprises enzymatically Hydrolyzed Reduced Minerals Whey Protein Concentrate, Vegetable Oils, Lactose, Corn Maltodextrin, and less than 1.5% of: Potassium Citrate, Potassium Phosphate, Calcium Chloride, Calcium Phosphate, Sodium Citrate, Magnesium Chloride, Ferrous Sulfate, Zinc Sulfate, Sodium Chloride, Copper Sulfate, Potassium Iodide, Manganese Sulfate, Vitamins, Taurine, Nucleotides, and L-Carnitine.

An additional aspect of the invention relates to compositions of the invention that are useful for increased intestinal absorption of a nutrient. The composition comprises a low-dose quantity of an enzyme selected from the group consisting of a lipase, an amylase, a protease, and any combination thereof. The composition is formulated for low-dose administration of the enzyme to a subject (e.g., an infant) in aqueous medium. In certain embodiments, the increase in intestinal absorption may be measured by an increase in height and/or weight, prevention of weight loss and/or normalization of protein stores as assessed by vitamins and albumin levels tested in subjects. In addition, the increase in intestinal absorption may be measured by a decrease in a symptom of pancreatic insufficiency or pancreatic malabsorption, e.g., bloating, colic, and/or diarrhea. Measurements of malabsorption may be made in stool samples (of fecal albumin level, e.g., 72-hour fecal fat measurements).

In another aspect, the invention is directed to a digestion enhancement composition. The composition comprises a low-dose quantity of an enzyme selected from the group consisting of a lipase, an amylase, a protease, and any combination thereof, and is formulated for low-dose administration of the enzyme to a subject (i.e., an infant) in aqueous medium. It would be understood by the ordinarily skilled artisan that digestion has a different measuring point than intestinal absorption. In fact, digestion may be measured by sampling the contents of the gastrointestinal tract. Alternatively, digestion may be similarly qualitatively measured by empirical improvement in the signs and symptoms of incomplete digestion.

In a further embodiment, the invention pertains to a packaged infant formula additive. This package comprises an enzyme selected from the group consisting of a lipase, an amylase, a protease, and any combination thereof, formulated for sustained stability in infant formula, as well as instructions for mixing the additive with infant formula and administration of the resulting product mixture to an infant.

Such packaged compositions are not intended to be limited solely to the administration to infants, but rather they are intended to have utility in all subjects, e.g., human subjects. Therefore, in yet another embodiment, the invention relates to a packaged additive comprising an enzyme selected from the group consisting of a lipase, an amylase, a protease, and any combination thereof, formulated for sustained stability in an aqueous medium, as well as instructions for mixing the additive with the aqueous medium and administration of the resulting product mixture to a subject.

Moreover, it is known that pasteurizing or boiling donor-expressed breast milk may reduce the absorption of fat to about 70% and about 65%, respectively, compared with natural human milk. In addition, human milk may be produced with a deficiency in the amount enzymes needed, for example, for fat absorption. Accordingly, the compositions of the invention may further be useful as an additive to human breast milk. As such, one embodiment of the invention is directed to a packaged breast milk additive comprising an enzyme selected from the group consisting of a lipase, an amylase, a protease, and any combination thereof, formulated for sustained stability in infant formula, as well as instructions for mixing the additive with breast milk and administration of the resulting product mixture to an infant.

The present invention further provides for the incorporation of encapsulated enzyme crystals or crystal formulations into the compositions of the present invention. Accordingly, enzyme crystals or crystal formulations are encapsulated within a matrix comprising a polymeric carrier to form a composition. The formulations and compositions enhance preservation of the native biologically active tertiary structure of the enzymes and create a reservoir which can slowly release active enzyme where and when it is needed. Such polymeric carriers include biocompatible and biodegradable polymers. The biologically active enzyme is subsequently released in a controlled manner over a period of time, as determined by the particular encapsulation technique, polymer formulation, crystal geometry, crystal solubility, crystal cross-linking and formulation conditions used. Methods are described herein for crystallizing enzymes, preparing stabilized formulations using pharmaceutical ingredients or excipients and optionally encapsulating them in a polymeric carrier to produce compositions and using such enzyme crystal formulations and compositions for biomedical applications, including delivery of therapeutic enzymes and vaccines.

The enzyme crystals may be combined with any conventional materials used for controlled release administration, including pharmaceutical controlled release administration. Such materials include, for example, coatings, shells and films, such as enteric coatings and polymer coatings and films.

Encapsulation of enzyme crystals or enzyme crystal formulations in polymeric carriers to make compositions may be carried out on enzyme crystals that are cross-linked or uncross-linked. Such enzyme crystals may be obtained commercially or produced as illustrated herein.

In addition, the amount of the enzyme or crystal formulations that provides a single dosage in a food product composition of the invention will vary depending upon the formulation itself, and dose level or dose frequency. A typical preparation will contain between about 0.01% and about 99%, preferably between about 1% and about 50%, enzyme crystals (w/w). Alternatively, a preparation will contain between about 0.01% and about 80% enzyme crystals, preferably between about 1% and about 50%, enzyme crystals (w/w). Alternatively, a preparation will contain between about 0.01% and about 80% enzyme crystal formulation, preferably between about 1% and about 50%, enzyme crystal formulation (w/w).

Upon improvement of the subject's condition, a maintenance dose of enzyme crystals or crystal formulations may be administered through use of the compositions of the invention, if necessary. Subsequently, the dosage or frequency of administration, or both, may be reduced as a function of the symptoms, to a level at which the improved condition is retained. When the condition has been alleviated to the desired level, treatment may cease. Individuals may, however, require intermittent treatment on a long-term basis upon any recurrence of the condition or symptoms thereof.

### III. Methods of the Invention

The invention is directed to a method of increasing intestinal absorption of a nutrient. The method comprises administering to an infant an enzyme selected from the group consisting of a lipase, an amylase, a protease, and any combination thereof, which is formulated for sustained stability in an aqueous medium. Moreover, the enzyme is also adapted for administration to an infant in the aqueous medium (e.g., infant formula), such that the intestinal absorption of the nutrient in the infant is increased.

The invention also pertains to a method of increasing intestinal absorption of a nutrient. The method comprises administering to a subject a low-dose quantity of an enzyme selected from the group consisting of a lipase, an amylase, a protease, and any combination thereof, which is adapted for administration to a subject (i.e., an infant) in an aqueous medium (e.g., infant formula). Furthermore, the enzyme is formulated for low-dose administration of the enzyme, such that the intestinal absorption of the nutrient in the subject is increased.

In yet another embodiment, the invention relates to a method of increasing food digestion. The method comprises administering to an infant an enzyme selected from the group consisting of a lipase, an amylase, a protease, and any combination thereof, where the enzyme is adapted for administration to an infant in an aqueous medium (e.g., infant formula), such that the digestion of food ingested by the infant is increased.

The invention includes a method of increasing food digestion. The method comprises administering to a subject a low-dose quantity of an enzyme selected from the group consisting of a lipase, an amylase, a protease, and any combination thereof, which is adapted for administration to a subject (i.e., an infant) in an aqueous medium (e.g., infant formula). Moreover, the enzyme is formulated for low-dose administration of the enzyme, such that the digestion of food ingested by the subject is increased.

### IV Methods of Preparation of Compositions of the Invention

### A. Production of Crystals and Crystal Formulations:

The enzyme crystal formulations useful in the present invention, which include microparticulate-based sustained release systems for enzyme drugs, advantageously permit improved patient compliance and convenience, more stable blood levels and potential dose reduction. The slow and constant release capabilities advantageously permit reduced dosages, due to more efficient delivery of active enzyme. Significant cost savings may be achieved by using the enzyme formulations and compositions described herein.

According to the one embodiment, crystal formulations for use in the food product compositions of the invention may prepared by the following process: first, the enzyme is crystallized. Next, excipients or ingredients selected from sugars, sugar alcohols, viscosity increasing agents, wetting or solubilizing agents, buffer salts, emulsifying agents, antimicrobial agents, antioxidants, and coating agents are added directly to the mother liquor. Alternatively, the crystals are suspended in an excipient solution, after the mother liquor is removed, for a minimum of 1 hour to a maximum of 24 hours. The excipient concentration is typically between about 0.01 to 30% W/W, which corresponds to a crystal concentration of 99.99 to 70% W/W, respectively. Most preferably, the excipient concentration is between about 0.1 to 10%, which corresponds to a crystal concentration of 99.9 to 90% W/W, respectively. The ingredient concentration is between about 0.01 to 90%. The crystal concentration is between about 0.01 to 95%. The mother liquor is then removed from the crystal slurry either by filtration or by centrifugation. Subsequently, the crystals are washed optionally with solutions of 50 to 100% one or more organic solvents such as, for example, ethanol, methanol, isopropanol or ethyl acetate, either at room temperature or at temperatures between −20° C. to 25° C. The crystals are the dried either by passing a stream of nitrogen, air, or inert gas over the crystals. Alternatively, the crystals are dried by air drying or by lyophilization or by vacuum drying. The drying is carried out for a minimum 1 hour to a maximum of 72 hours after washing, until the moisture content of the final product is below 10% by weight, most preferably below 5%. Finally, micronizing of the crystals can be performed if necessary.

When preparing enzyme crystals, enzyme crystal formulations, enhancers, such as surfactants often are not added during crystallization. Excipients or ingredients are added to the mother liquor after crystallization, at a concentration of between about 1-10% W/W, alternatively at a concentration of between about 0.1-25% W/W, alternatively at a concentration of between about 0.1-50% W/W. These concentrations correspond to crystal concentrations of 99-90% W/W, 99.9-75% W/W and 99.9-50% W/W, respectively. The excipient or ingredient is incubated with the crystals in the mother liquor for about 0.1-3 hrs, alternatively the incubation is carried out for 0.1-12 hrs, alternatively the incubation is carried out for 0.1-24 hrs. The ingredient or excipient may be dissolved in a solution other than the mother liquor, and the enzyme crystals are removed from the mother liquor and suspended in the excipient or ingredient solution. The ingredient or excipient concentrations and the incubation times are the same as those described above.

### B. Slow Release Forms

In another embodiment of this invention, the food product compositions may incorporate lipases that are encapsulated in polymeric carriers. The flexibility in preparing slowly available active lipase solves the problems often associated with lipase supplementation. As such, the food product compositions of the present invention may include the combination of encapsulated lipase crystals and unencapsulated cross-linked lipase crystals or formulations to provide a therapy regime in which enzyme activity is available early on from the unencapsulated cross-linked lipase. As this material undergoes proteolytic degradation, the encapsulated enzyme begins to release enzyme activity into the more distal bowel.

The enzyme crystals encapsulated within polymeric carriers, and forming a composition comprising microspheres, can also be dried by lyophilization. Lyophilization, or freeze-drying, allows water to be separated from the composition.

The enzyme crystal formulation is first frozen and then placed in a high vacuum. In a vacuum, the crystalline H₂O sublimes, leaving the enzyme crystal composition behind containing only the tightly bound water. Such processing further stabilizes the composition and allows for easier storage and transportation at typically encountered ambient temperatures.

### C. Enzyme Crystallization

Enzyme crystals may be grown by controlled crystallization of enzyme from aqueous solutions or aqueous solutions containing organic solvents. Solution conditions that may be controlled include, for example, the rate of evaporation of solvent, organic solvents, the presence of appropriate co-solutes and buffers, pH and temperature. A comprehensive review of the various factors affecting the crystallization of enzymes has been published by McPherson, Methods Enzymol., 114, pp. 112-20 (1985).

McPherson and Gilliland, J. Crystal Growth, 90, pp. 51-59 (1988) have compiled comprehensive lists of enzymes that have been crystallized, as well as the conditions under which they were crystallized. A compendium of crystals and crystallization recipes, as well as a repository of coordinates of solved enzyme structures, is maintained by the Protein Data Bank at the Brookhaven National Laboratory [http//www. pdb.bnl.gov; Bernstein et al., J. Mol. Biol., 112, pp. 535-42 (1977)]. These references can be used to determine the conditions necessary for crystallization of an enzyme, as a prelude to the formation of appropriate enzyme crystals and can guide the crystallization strategy for other enzymes. Alternatively, an intelligent trial and error search strategy can, in most instances, produce suitable crystallization conditions for many enzymes, provided that an acceptable level of purity can be achieved for them [see, e.g., C. W. Carter, Jr. and C. W. Carter, J. Biol. Chem., 254, pp. 12219-23 (1979)].

In general, crystals are produced by combining the enzyme to be crystallized with an appropriate aqueous solvent or aqueous solvent containing appropriate crystallization agents, such as salts or organic solvents. The solvent is combined with the enzyme and may be subjected to agitation at a temperature determined experimentally to be appropriate for the induction of crystallization and acceptable for the maintenance of enzyme activity and stability. The solvent can optionally include co-solutes, such as divalent cations, co-factors or chaotropes, as well as buffer species to control pH. The need for co-solutes and their concentrations are determined experimentally to facilitate crystallization.

In an industrial-scale process, the controlled precipitation leading to crystallization can best be carried out by the simple combination of enzyme, precipitant, co-solutes and, optionally, buffers in a batch process. As another option, enzymes may be crystallized by using enzyme precipitates as the starting material. In this case, enzyme precipitates are added to a crystallization solution and incubated until crystals form. Alternative laboratory crystallization methods, such as dialysis or vapor diffusion, can also be adopted. McPherson, supra and Gilliland, supra, include a comprehensive list of suitable conditions in their reviews of the crystallization literature.

Occasionally, in cases in which the crystallized enzyme is to be cross-linked, incompatibility between an intended cross-linking agent and the crystallization medium might require exchanging the crystals into a more suitable solvent system.

### D. Cross-Linking of Enzyme Crystals

The release rate of the enzyme from the polymeric carrier may be slowed and controlled by using enzyme crystals that have been chemically cross-linked using a cross-linker, such as for example, a biocompatible cross-linker. Thus, once enzyme crystals have been grown in a suitable medium they may be cross-linked.

Cross-linking may be carried out using reversible cross-linkers, in parallel or in sequence. The resulting cross-linked enzyme crystals are characterized by a reactive multi-functional linker, into which a trigger is incorporated as a separate group. The reactive functionality is involved in linking together reactive amino acid side chains in an enzyme and the trigger consists of a bond that can be broken by altering one or more conditions in the surrounding environment (e.g., pH, temperature, or thermodynamic water activity).

The bond between the cross-linking agent and the enzyme may be a covalent or ionic bond, or a hydrogen bond. The change in surrounding environment results in breaking of the trigger bond and dissolution of the enzyme. Thus, when the cross-links within enzyme crystals cross-linked with such reversible cross-linking agents break, dissolution of enzyme crystal begins and therefore the release of activity.

Alternatively, the reactive functionality of the cross-linker and the trigger may be the same.

The cross-linker may be homofunctional or heterofunctional , and are further described in U.S. Pat. No. 6,541,606, the contents of which have already been incorporated by reference herein. For example, the reactive groups can be any variety of groups such as those susceptible to nucleophilic, free radical or electrophilic displacement including halides, aldehydes, carbonates, urethanes, xanthanes, epoxides among others.

### E. Encapsulation of Enzyme Crystals in Polymeric Carriers

The enzyme crystals may be encapsulated in at least one polymeric carrier to form microspheres by virtue of encapsulation within the matrix of the polymeric carrier to preserve their native and biologically active tertiary structure. The crystals can be encapsulated using various biocompatible and/or biodegradable polymers having unique properties that are suitable for delivery to different biological environments or for effecting specific functions. The rate of dissolution and, therefore, delivery of active enzyme is determined by the particular encapsulation technique, polymer composition, polymer cross-linking, polymer thickness, polymer solubility, enzyme crystal geometry and degree and, if any, of enzyme crystal cross-linking

Enzyme crystals or formulations to be encapsulated are suspended in a polymeric carrier that is dissolved in an organic solvent. The polymer solution must be concentrated enough to completely coat the enzyme crystals or formulations after they are added to the solution. Such an amount is one that provides a weight ratio of enzyme crystals to polymer between about 0.02 and about 20, preferably between about 0.1 and about 2. The enzyme crystals are contacted with polymer in solution for a period of time between about 0.5 minutes and about 30 minutes, preferably between about 1 minute and about 3 minutes. The crystals should be kept suspended and not allowed to aggregate as they are coated by contact with the polymer.

Following that contact, the crystals become coated and are referred to as nascent microspheres. The nascent microspheres increase in size while coating occurs. In a preferred embodiment, the suspended coated crystals or nascent microspheres along with the polymeric carrier and organic solvent are transferred to a larger volume of an aqueous solution containing a surface active agent, known as an emulsifier. In the aqueous solution, the suspended nascent microspheres are immersed in the aqueous phase, where the organic solvent evaporates or diffuses away from the polymer. Eventually, a point is reached where the polymer is no longer soluble and forms a precipitated phase encapsulating the enzyme crystals or formulations to form a composition. This aspect of the process is referred to as hardening of the polymeric carrier or polymer. The emulsifier helps to reduce the interfacial surface tension between the various phases of matter in the system during the hardening phase of the process. Alternatively, if the coating polymer has some inherent surface activity, there may be no need for addition of a separate surface active agent.

Emulsifiers useful to prepare encapsulated enzyme crystals useful in the compositions of the present invention include poly(vinyl alcohol) as exemplified herein, surfactants and other surface active agents which can reduce the surface tension between the polymer coated enzyme crystals or polymer coated crystal formulations and the solution.

Organic solvents useful to prepare the microspheres useful in the compositions of the present invention include methylene chloride, ethyl acetate, chloroform and other non-toxic solvents, which will depend on the properties of the polymer. Solvents should be chosen that solubilize the polymer and are ultimately non-toxic.

The crystallinity of the enzyme crystals is preferably maintained during the encapsulation process. The crystallinity may be maintained during the coating process by using an organic solvent in which the crystals are not soluble. Subsequently, once the coated crystals are transferred to the aqueous solvent, rapid hardening of the polymeric carrier and sufficient coating of the crystals in the previous step shields the crystalline material from dissolution. In another embodiment, the use of cross-linked enzyme crystals facilitates maintenance of crystallinity in both the aqueous and organic solvents.

The polymers used as polymeric carriers to coat the enzyme crystals can be either homo-polymers or co-polymers. The rate of hydrolysis of the microspheres is largely determined by the hydrolysis rate of the individual polymer species. In general, the rate of hydrolysis decreases as follows: polycarbonates>polyesters>polyurethanes>polyorthoesters>polyamides. For a review of biodegradable and biocompatible polymers, see W. R. Gombotz and D. K. Pettit, “Biodegradable polymers for enzyme and peptide drug delivery”, Bioconjugate Chemistry, vol. 6, pp. 332-351 (1995).

In a preferred embodiment, the polymeric carrier is composed of a single polymer type such as PLGA. The polymeric carrier can also be a mixture of polymers such as 50% PLGA and 50% albumin.

Other polymers useful as polymeric carriers to prepare encapsulated enzyme crystals include biocompatinie/biodegradable polymers selected from the group consisting of poly (acrylic acid), poly (cyanoacrylates), poly (amino acids), poly (anhydrides), poly (depsipeptide), poly (esters), such as poly (lactic acid) or PLA, poly (b-hydroxybutryate), poly (caprolactone) and poly (dioxanone); poly (ethylene glycol), poly (hydroxypropyl)methacrylamide, poly [(organo)phosphazene], poly (ortho esters), poly (vinyl alcohol), poly (vinylpyrrolidone), maleic anhydride-alkyl vinyl ether copolymers, pluronic polyols, albumin, alginate, cellulose and cellulose derivatives, collagen, fibrin, gelatin, hyaluronic acid, oligosaccharides, glycaminoglycans, sulfated polysaccharides, blends and copolymers thereof. Other useful polymers are described in J. Heller and R. W. Balar, “Theory and Practice of Controlled Drug Delivery from Biodegradable Polymers,” Academic Press, New York, N.Y., (1980); K. O. R. Lehman and D. K. Dreher, Pharmaceutical Technology, vol. 3, pp. 5, (1979); E. M. Ramadan, A. El-Helw and Y. El-Said, Journal of Microencapsulation, vol. 5, p. 125 (1988). The preferred polymer will depend upon the particular enzyme component of the enzyme crystals used and the intended use of the encapsulated crystals (formulations and compositions). Alternatively, the solvent evaporation technique may be used for encapsulating enzyme crystals (see D. Babay, A. Hoffmann and S. Benita, Biomaterials vol. 9, pp. 482-488 (1988).

Enzyme crystals are preferably encapsulated in at least one polymeric carrier using a double emulsion method, as illustrated herein, using a polymer, such as polylactic-co-glycolyic acid. In a most preferred embodiment, the polymer is polylactic-co-glycolyic acid (“PLGA”). PLGA is a co-polymer prepared by polycondensation reactions with lactic acid (“L”) and glycolic acid (“G”). Various ratios of L and G can be used to modulate the crystallinity and hydrophobicity of the PLGA polymer. Higher crystallinity of the polymer results in slower dissolution. PLGA polymers with 20-70% G content tend to be amorphous solids, while high level of either G or L result in good polymer crystallinity. For more information on preparing PLGA, see D. K. Gilding and A. M. Reed, “Biodegradable polymers for use in surgery-poly(glycolic)/poly(lactic acid) homo and copolymers: 1., Polymer vol. 20, pp. 1459-1464 (1981). PLGA degrades after exposure to water by hydrolysis of the ester bond linkage to yield non-toxic monomers of lactic acid and glycolic acid.

In another embodiment, double-walled polymer coated microspheres may be advantageous. Double-walled polymer coated microspheres may be produced by preparing two separate polymer solutions in methylene chloride or other solvent which can dissolve the polymers. The enzyme crystals are added to one of the solutions and dispersed. Here, the enzyme crystals become coated with the first polymer. Then, the solution containing the first polymer coated enzyme crystals is combined with the second polymer solution. [See Pekarek, K. J.; Jacob, J. S. and Mathiowitz, E. Double-walled polymer microspheres for controlled drug release, Nature, 367, 258-260]. Now, the second polymer encapsulates the first polymer which is encapsulating the enzyme crystal. Ideally, this solution is then dripped into a larger volume of an aqueous solution containing a surface active agent or emulsifier. In the aqueous solution, the solvent evaporates from the two polymer solutions and the polymers are precipitated.

Formulations of the enzyme crystals useful in the compositions of the invention may comprise an enzyme crystal, and at least one ingredient. Such enzyme crystal formulations may be characterized by at least a 60 fold greater shelf life when stored at 50° C. than the soluble form of said enzyme in solution at 50° C., as measured by T_{1/2}. Alternatively, they may be characterized by at least a 59 fold greater shelf life when stored at 40° C. and 75% humidity than the nonformulated form of said enzyme crystal when stored at 40° C. and 75% humidity, as measured by T_{1/2}. They may also be characterized by at least a 60% greater shelf life when stored at 50° C. than the nonformulated form of said enzyme crystal when stored at 50° C., as measured by T_{1/2}. Similarly, they may be characterized by the loss of less than 20% α-helical structural content of the enzyme after storage for 4 days at 50° C., wherein the soluble form of said enzyme loses more than 50% of its α-helical structural content after storage for 6 hours at 50° C. as measured by FTIR; or by the loss of less than 20% α-helical structural content of the enzyme after storage for 4 days at 50° C., wherein the soluble form of said enzyme loses more than 50% of its .alpha.-helical structural content after storage for 6 hours at 50° C. as measured by FTIR, and wherein said formulation is characterized by at least a 60 fold greater shelf life when stored at 50° C. than the soluble form of said enzyme in solution at 50° C., as measured by T_{1/2}.

In addition, compositions according to this invention may comprise one of the above described enzyme crystal formulations, and, at least one polymeric carrier, wherein said formulation is encapsulated within a matrix of said polymeric carrier.

In order that this invention may be better understood, the following examples are set forth. These examples are for the purpose of illustration only and are not to be construed as limiting the scope of the invention in any manner.

### EXEMPLIFICATION

The compositions described herein relate to food products, i.e., nutritional food products and instant formula, and explicitly contain at least one lipase, protease or amylase enzyme that may be formulated to have sustained stability in an aqueous medium.

The stability of the enzyme to be used in the food products of the invention derives from the preparation of the protein/enzyme itself or its combination with an excipient. Accordingly, the following examples depict several techniques that may be used to prepare such enzymes for inclusion in the food products of the present invention.

However, while the following examples may be useful in describing the methods of making certain enzymes, and the compositions containing same, these examples are not intended to be limiting of the invention. In fact, many of these examples may be more completely described in U.S. Pat. No. 6,541,606, which has already been incorporated by reference herein in its entirety (inclusive of the Exemplification Section).

The food product compositions of the present invention may be made using any technique known to the ordinarily skilled artisan. Exemplary compositions to which an enzyme selected from lipase, protease, or amylase enzymes may be added would be compatible in miscibility to the enzymes, substantially inert to the enzymes (i.e., the components of the composition would be essentially non-reactive with the enzymes), and the techniques in manufacturing to produce a final food product composition of the present invention would not significantly affect the form or stability of the enzyme as to be deleterious to the purposes of the invention.

Moreover, the present invention includes the addition of the lipase, protease, or amylase enzymes, for example as prepared below, to existing commercially available products, such as Good Start Supreme, Good Start 2 with DHA and ARA, Good Start 2 Soy DHA and ARA, Good Start Supreme DHA and ARA, and Good Start Essentials, manufactured by Nestlé.

### EXAMPLE 1

Candida rugosa Lipase Crystallization

### Materials

(A) Candida rugosa lipase powder

(B) Celite powder (diatomite earth)

(C) MPD (2-Methyl-2,4-Pentanediol)

(D) 5 mM Ca acetate buffer pH 4.6

(E) Deionized water

### Procedure:

A 1 kg aliquot of lipase powder is mixed well with 1 kg of celite and then 22 L of distilled water is added. The mixture is stirred to dissolve the lipase powder. After dissolution is complete, the pH is adjusted to 4.8 using acetic acid. Next, the solution is filtered to remove celite and undissolved materials. Then, the filtrate is pumped through a 30 k cut-off hollow fiber to remove all the proteins that are less than 30 kD molecular weight. Distilled water is added and the lipase filtrate is pumped through the hollow fiber until the retentate conductivity was equal to the conductivity of the distilled water. At this point, the addition of distilled water is stopped and 5 mm Ca-acetate buffer is added. Next, Ca-acetate buffer is delivered by pumping through the hollow fiber until the conductivity of the retentate is equal to the conductivity of the Ca-acetate buffer. At that point, addition of the buffer is stopped. The lipase solution is concentrated to 30 mg/ml solution. The crystallization is initiated by pumping MPD slowly into the lipase solution while stirring. Addition of MPD is continued until a 20% vol/vol of MPD is reached. The mixture is stirred for 24 hr or until 90% of the protein crystallizes. The resulting crystals are washed with crystallization buffer to remove all the soluble material from the crystals. Then, the crystals are suspended in fresh crystallization buffer to achieve a protein concentration of 42 mg/ml.

### EXAMPLE 2

### Formulation of Lipase Crystals Using Sucrose as Excipient

In order to enhance the stability of lipase crystals during drying and storage the crystals may be formulated with excipients. In this example, lipase crystals are formulated in the slurry form in the presence of mother liquor before drying. Sucrose (Sigma Chemical Co., St. Louis, Mo.) is added to lipase crystals in mother liquor as an excipient. Sufficient sucrose is added to lipase crystals at a protein concentration of 20 mgs/ml in mother liquor (10 mM sodium acetate buffer, pH 4.8 containing 10 mM Calcium chloride and 20% MPD) to reach a final concentration of 10%. The resulting suspension is tumbled at room temperature for 3 hr. After treatment with sucrose, the crystals are separated from the liquid by centrifugation as described in Example 6, method 4 or 5.

### EXAMPLE 3

### Formulation of Lipase Crystals Using Trehalose as Excipient

The lipase crystals are formulated as in Example 2, by adding trehalose, instead of sucrose, (Sigma Chemical Co., St. Louis, Mo.), to a final concentration of 10% in mother liquor. The resulting suspension is tumbled at room temperature for 3 hr and the crystals are separated from the liquid by centrifugation as described in Example 6, method 4 or 5.

### EXAMPLE 4

### Formulation of Lipase Crystals Using Polyethylene Oxide (PEO) as Excipient

Lipase crystals may be formulated using 0.1% polyethylene oxide in water as follows. The crystals, in the mother liquor at 20 mg/ml are separated from the mother liquor by centrifugation at 1000 rpm in a Beckman GS-6R bench top centrifuge equipped with swinging bucket rotor. Next, the crystals are suspended in 0.1% polyethylene oxide for 3 hrs (Sigma Chemical Co., St. Louis, Mo.) and then separated by centrifugation, as described in Example 6, method 4 or 5.

### EXAMPLE 5

### Formulation of Lipase Crystals Using Methoxypolyethylene Glycol (MOPEG) as Excipient

Lipase crystals may be formulated as in Example 2, by adding 10% methoxypoly ethylene glycol, instead of sucrose, (final concentration) (Sigma Chemical Co., St. Louis, Mo.) in mother liquor and separating after 3 hrs by centrifugation, as in Example 6, method 4 or 5.

### EXAMPLE 6

### Methods of Drying Crystal Formulation

### A. Method 1: N₂ Gas Drying at Room Temperature

Crystals as prepared in Example 1 are separated from the mother liquor containing excipient by centrifugation at 1000 rpm in a Beckman GS-6R bench top centrifuge equipped with swinging bucket rotor in a 50 ml Fisher brand Disposable centrifuge tube (Polypropylene). The crystals are then dried by passing a stream of nitrogen at approximately 10 psi pressure into the tube overnight.

### B. Method 2: Vacuum Oven Drying

Crystals as prepared in Example 1 are first separated from the mother liquor/excipient solution using centrifugation at 1000 rpm in a Beckman GS-6R bench top centrifuge equipped with swinging bucket rotor in a 50 ml Fisher brand

Disposable polypropylene centrifuge tube. The wet crystals are then placed in a vacuum oven at 25 in Hg (VWR Scientific Products) at room temperature and dried for at least 12 hours.

### C. Method 3: Lyophilization

Crystals as prepared in Example are first separated from the mother liquor/excipient solution using centrifugation at 1000 rpm in a Beckman GS-6R bench top centrifuge equipped with swinging bucket rotor in a 50 ml Fisher brand Disposable polypropylene centrifuge tube. The wet crystals are then freeze dried using a Virtis Lyophilizer Model 24 in semistoppered vials. The shelf temperature is slowly reduced to −40° C. during the freezing step. This temperature is held for 16 hrs. Secondary drying is then carried out for another 8 hrs.

### D. Method 4: Organic Solvent and Air Drying

Crystals as prepared in Example 1 are first separated from the mother liquor/excipient solution using centrifugation at 1000 rpm in a Beckman GS-6R bench top centrifuge equipped with swinging bucket rotor in a 50 ml Fisher brand Disposable polypropylene centrifuge tube. The crystals are then suspended in an organic solvent like ethanol or isopropanol or ethyl acetate or other suitable solvents, centrifuged, the supernatant is decanted and air dried at room temperature in the fume hood for two days.

### E. Method 5: Air Drying at Room Temperature

Crystals as prepared in Example 1 are separated from the mother liquor containing excipient by centrifugation at 1000 rpm in a Beckman GS-6R bench top centrifuge equipped with swinging bucket rotor in a 50 ml Fisher brand Disposable centrifuge tube (Polypropylene). Subsequently, the crystals are allowed to air dry in the fume hood for two days.

### EXAMPLE 7

### Soluble Lipase Sample Preparation

For comparison, a sample of soluble lipase was prepared by dissolving lipase crystals to 20 mg/ml in phosphate buffered saline, pH 7.4. Stability, specific activity and the T_{1/2} for soluble lipase is then calculated.

### EXAMPLE 8

### Olive Oil Assay for Measuring Lipase Activity

Lipase crystals may be assessed for activity against olive oil in pH 7.7 buffer. The assay is carried out titrimetrically using slight modifications to the procedure described in Pharmaceutical Enzymes—Properties and Assay Methods, R. Ruyssen and A. Lauwers, (Eds.), Scientific Publishing Company, Ghent, Belgium (1978).

### Reagents:

(1) Olive oil emulsion: 16.5 gm of gum arabic (Sigma) is dissolved in 180 ml of water, 20 ml of olive oil (Sigma) and emulsified using a Quick Prep mixer for 3 minutes. (2) Titrant : 0.05 M NaOH. (3) Solution A: 3.0 M NaCl (4) Solution B: 75 mM CaCl₂-2H2O (5) Mix: 40 ml of Solution A was combined with 20 ml of Solution B and 100 ml of H2 O. (6) 0.5% Albumin. (7) Lipase Substrate Solution (solution 7) is prepared by adding 50 ml of olive oil emulsion (solution 1) to 40 ml of Mix (solution 5) and 10 ml of 0.5% albumin (solution 6).

### Assay Procedure:

The lipase substrate solution (solution 7) is warmed to 37° C. in a water bath. First, 20 ml of substrate is added to a reaction vessel and the pH is adjusted to 7.7 using 0.05 M NaOH (solution 2) and equilibrated to 37° C. with stirring. The reaction is initiated by adding enzyme. The reaction progress is monitored by titrating the mixture of enzyme and substrate with 0.05 M NaOH to maintain the pH at 7.7.

The specific activity (moles/min/mg protein) is equal to the initial rate×1000×concentration of the titrant/the amount of enzyme. The zero point is determined by running the reaction without enzyme, i.e., using buffer in the place of enzyme in the reaction mixture.

### EXAMPLE 9

### Shelf Activity of the Dried Crystals

### Activity:

The shelf activity of the dried crystals from Examples 1-5 may be measured using the olive oil assay as described in Example 8. Dried crystals (5 mg) are dissolved in 1 ml of phosphate buffered saline (“PBS”), pH 7.4 and the activity is measured using olive oil as substrate.

### Shelf Stability:

The shelf stability of dried crystalline lipase formulations from Examples 2-5 may carried out in a humidity chamber controlled at 75% relative humidity and 40° C. temperature (HOTPACK). The activity of the crystals is measured by dissolving 5 mg of the dried samples in PBS buffer, pH 7.4, measuring the activity in the olive oil assay and then comparing with the initial results.

The T_{1/2} may be calculated from the shelf life data by non-linear regression analysis using the Sigma Plot program.

### Moisture Content:

Moisture content may be determined by the Karl Fischer method according to manufacturer's instructions using a Mitsubishi CA-06 Moisture Meter equipped with a VA-06 Vaporizer (Mitsubishi Chemical Corporation, Tokyo, Japan).

### Crystallinity:

The crystal integrity of the formulations may be measured by quantitative microscopic observations. In order to visualize whether the crystals maintain their shape after drying, the dried crystals are examined under an Olympus BX60 microscope equipped with DXC-970MD 3CCD Color Video Camera with Camera Adapter (CMA D2) with Image ProPlus software. Samples of dried crystals are covered with a glass coverslip, mounted and examined under 10.times. magnification, using an Olympus microscope with an Olympus UPLAN Fl objective lens 10×/0.30 PH1 (phase contrast).

### Secondary Structure Characterization by FTIR:

The Fourier transform infrared (“FTIR”) spectra may be collected on a Nicolet model 550 Magna series spectrometer as described by Dong et al. [Dong, A., Caughey, B., Caughey, W. S., Bhat, K. S. and Coe, J. E. Biochemistry, 1992; 31:9364-9370; Dong, A. Prestrelski, S. J., Allison, S. D. and Carpenter, J. F. J. Pharm. Sci., 1995; 84: 415-424.] For the solid samples, 1 to 2 mg of the protein is lightly ground with 350 mg of KBr powder and filled into small cups used for diffuse reflectance accessory. The spectra are collected and then processed using Grams 32 from Galactic software for the determination of relative areas of the individual components of secondary structure using second derivative and curve-fitting program under amide I region (1600-1700 cm⁻¹).

For comparison, a soluble lipase sample may be prepared by dissolving lipase crystals in phosphate buffered saline and analyzed for stability by FTIR.

Secondary structure may be determined as follows: FTIR spectra are collected on a Nicolet model 550 Magna series spectrometer. A 1 ml sample of soluble lipase is placed on a Zinc selenide crystal of ARK ESP. The spectra are collected at initial (0) time and after the loss of most of the activity or, near-zero activity. The acquired data is then processed using Grams 32 software from Galactic Software for the determination of relative areas of the individual components of secondary structure using second derivative and curve-fitting program under amide I region (1600-1700 cm⁻¹).

### EXAMPLE 10

Drying of Candida rugosa Lipase Crystals

### Materials:

(A) Candida rugosa lipase (Example 1)

(B) Poly(ethylene glycol), 100% PEG 200, 300, 400, or 600

(C) Acetone

### Procedure:

A 4 ml aliquot of crystal suspension (140 mg) is added to four 15 ml tubes. Next, the suspension is centrifuged at between 1000 to 3000 RPM for between 1 to 5 minutes or until the crystallization buffer is removed. Then, 4 ml of liquid polymer (any PEG between 200 to 600 is suitable) is added to each tube and the contents are mixed until homogeneous. The suspension is centrifuged at between 1000 to 3000 RPM for between 1 to 5 minutes or until the liquid polymer is removed. Next, 4 ml of acetone (isopropanol, butanol and other solvents are also suitable) is added to each tube and mixed well. The crystal/organic solvent suspensions are transferred to 0.8 cm×4 cm BIO-RAD poly-prep chromatography columns (spin columns). The columns are centrifuged at 1000 RPM for 1 to 5 minutes to remove the organic solvent.

Finally, nitrogen gas is passed through the column to dry the crystals until a free flowing powder results.

### EXAMPLE 11

### Purafect (Protease) 4000 L Crystallization

### Materials:

(A) Crude purafect 4000 L (protease enzyme)

(B) 15% Na₂SO₄ solution

### Procedure:

One volume of crude purafect enzyme solution is mixed with two volumes of 15% Na₂SO₄ solution. The mixture is stirred for 24 hr at room temperature or until the crystallization is completed. The crystals are washed with 15% Na₂SO₄ solution to eliminate the soluble enzyme. The crystals are suspended in fresh 15% Na₂SO₄ solution to yield a protein concentration of 27 mg/ml.

### EXAMPLE 12

### Drying of Purafect Crystals

### Materials:

(A) Purafect crystals suspension (protease)

(B) Poly(ethylene glycol), 100% PEG 200, 300, 400, or 600

(C) Organic solution

### Procedure:

A 4 ml aliquot of crystal suspension (140 mg) is added to four 15 ml tubes. Next, the suspension is centrifuged at between 1000 to 3000 RPM for between 1 to 5 minutes or until the crystallization buffer is removed. Then, 4 ml of liquid polymer (any PEG between 200 to 600 is suitable) is added to each tube and the contents are mixed until homogeneous. The suspension is centrifuged at between 1000 to 3000

RPM for between 1 to 5 minutes or until the liquid polymer is removed. Next, 4 ml of acetone (isopropanol, butanol and other solvents are also suitable) is added to each tube and mixed well. The crystal/organic solvent suspensions are transferred to 0.8 cm×4 cm BIO-RAD poly-prep chromatography columns (spin columns). The columns are centrifuged at 1000 RPM for 1 to 5 minutes to remove the organic solvent.

Finally, nitrogen gas is passed through the column to dry the crystals until a free flowing powder results.

### EXAMPLE 13

Large Scale Crystallization of Pseudomonas cepacia Lipase

A slurry of 15 kg crude Pseudomonas cepacia lipase (PS 30 lipase-Amano) (“LPS”) is dissolved in 100 L distilled deionized water and the volume brought to 200 L with additional distilled deionized water. The suspension is mixed in an Air Drive Lightning mixer for 2 hours at room temperature and then filtered through a 0.5 μm filter to remove celite. The mixture is then ultrafiltered and concentrated to 10 L (121.4 g) using a 3K hollow fiber filter membrane cartridge. Solid calcium acetate is added to a concentration of 20 mM Ca(CH₃ COO)₂. The pH is adjusted to 5.5 with concentrated acetic acid, as necessary. The mixture is heated to and maintained at a temperature of 30° C. Magnesium sulfate is added to a 0.2 M concentration, followed by glucopon to a 1% concentration. Isopropanol is then added to a final concentration of 23%. The resulting solution is mixed for 30 minutes at 30° C., and then cooled from 30° C. to 12° C. over a 2-hour period. Crystallization is then allowed to proceed for 16 hours.

The crystals are allowed to settle and soluble protein is removed using a peristaltic pump with tygon tubing having a 10 ml pipette at its end. Fresh crystallization solution (23% isopropyl alcohol, 0.2 M MgSO₄, 1% glucopon, 20 mM Ca(CH₃ COO)₂, pH 5.5) is added to bring the concentration of protein to 30 mg/ml (O.D. 280 of a 1 mg/ml solution=1.0, measured using a spectrophotometer at wavelength 280). The crystal yield is then determined.

### EXAMPLE 14

### Cross-Linked LPS Crystals

Cross-linked Pseudomonas cepacia lipase crystals, sold under the name ChiroCLEC-PC™, are available from Altus Biologics, Inc. (Cambridge, Mass.) may be used to produce formulations according to Example 16. Alternatively, lipase crystals as prepared above may be cross-linked using any conventional method.

### EXAMPLE 15

Cross-Linked Candida rugosa Lipase Crystals

Cross-linked Candida rugosa lipase crystals, sold under the name ChiroCLEC-CR™, are available from Altus Biologics, Inc. (Cambridge, Mass.) and may be used to produce formulations according to Example 16. Alternatively, lipase crystals as prepared above, may be cross-linked using any conventional method.

### EXAMPLE 16

### Microencapsulation of Protein Crystals in Polylactic-co-glycolic Acid (PLGA)

Microencapsulation may be performed using uncross-linked crystals of lipase from Candida rugosa and Pseudomonas cepacia. Further, microencapsulation is performed using cross-linked enzyme crystals of lipase from Candida rugosa. The microencapsulation process results in microspheres. In addition, any other protein crystals or protein crystal formulation produced may be encapsulated by this technique.

### A. Preparation of Dry Crystals

Crystals or crystal formulations dried according to Example 6 may each be used to produce the microspheres for inclusion in the compositions of this invention. One process for drying protein crystals for use in the compositions of this invention involves air drying.

Approximately 500 mg each of Candida rugosa lipase crystals from Example 1 (uncross-linked and cross-linked) are air dried. First, the mother liquor is removed by centrifugation at 3000 rpm for 5 minutes. Next, the crystals are allowed to stand at 25° C. in the fume hood for two days.

### B. Polymer and Solvents

The polymer used to encapsulate the protein crystals was PLGA. PLGA was purchased as 50/50 Poly(DL-lactide-co-glycolide) from Birmingham Polymers, Inc. from Lot No. D97188. This lot had an inherent viscosity of 0.44 dl/g in HFIP@ 30° C.

The methylene chloride was spectroscopic grade and was purchased from Aldrich Chemical Co. Milwaukee, Wis. The poly vinyl alcohol was purchased from Aldrich Chemical Co. Milwaukee, Wis.

### D. Encapsulation of Crystals in PLGA

The crystals may be encapsulated in PLGA using a double emulsion method. The general process is as follows, either dry protein crystals or a slurry of protein crystals are first added to a polymer solution in methylene chloride. The crystals are coated with the polymer and become nascent microspheres. Next, the polymer in organic solvent solution is transferred to a much larger volume of an aqueous solution containing a surface active agent. As a result, the organic solvent began to evaporate and the polymer hardens. In this example, two successive aqueous solutions of decreasing concentrations of emulsifier are employed for hardening of the polymer coat to form microspheres.

The following procedure was one exemplification of this general process. Those of skill in the art of polymer science will appreciate that many variations of the procedure may be employed and the following example is not meant to limit the invention.

### Use of Dry Protein Crystals

Dry crystals of cross-linked and uncross-linked Candida rugosa lipase produced according to Example 1 are were weighed into 150 mg samples. The weighed protein crystals are then added directly into a 15 ml polypropylene centrifuge tube (Fisher Scientific) containing 2 ml of methylene chloride with PLGA at 0.6 g PLGA/ml solvent. The crystals are added directly to the surface of the solvent. Next, the tube is thoroughly mixed by vortexing for 2 minutes at room temperature to completely disperse the protein crystals in the solvent with PLGA. The crystals are allowed to become completely coated with polymer. Further vortexing or agitation may be used to keep the nascent microspheres suspended to allow further coating. The polymer may be hardened as described in section (iii)

### (ii) Use of a Protein Crystal Slurry

A crystal slurry of Pseudomonas cepacia lipase may be produced using approximately 50 mg of crystals per 200 μl of mother liquor. The crystal slurry is rapidly injected into a 15 ml polypropylene centrifuge tube (Fisher Scientific) with 2 ml of a solution of methylene chloride and poly(lactic-co-glycolic acid) at 0.6 g PLGA/ml solvent. The needle is inserted below the surface of the solvent and injected into the solution. In this case, 150 mg of total protein, or 600 μl of aqueous solution, is injected. The injection is made using a plastic syringe Leur-lok (Becton-Dickinson & Company) and through a 22 gauge (Becton-Dickinson & Company) stainless steel needle. Next, the protein crystal-PLGA slurry is mixed thoroughly by vortexing for 2 minutes at room temperature. The crystals are allowed to be completely coated with polymer. Further vortexing or agitation may be optionally used to keep the nascent microspheres suspended to allow further coating.

(iii) Hardening the Polymer Coating

A two step process may be employed to facilitate the removal of methylene chloride from the liquid polymer coat and allow the polymer to harden onto the protein crystals. The difference between the steps is that the concentration of emulsifier is much higher in the first solution and the volume of the first solution is much smaller than the second.

In the first step, the polymer coated crystal and methylene chloride suspension is added dropwise to a stirred flask of 180 ml of 6% polyvinyl alcohol (hereinafter “PVA”) in water with 0.5% methylene chloride at room temperature. This solution is mixed rapidly for 1 minute.

In step two, the first PVA solution containing the nascent microspheres is rapidly poured into 2.4 liters of cold (4° C.) distilled water. This final bath is mixed gently at 4° C. for 1 hr with the surface of the solution under nitrogen. After 1 hr, the microspheres are filtered using 0.22 μm filter and washed with 3 liters of distilled water containing 0.1% Tween 20 to reduce agglomeration.

### EXAMPLE 17

### Production of Encapsulated Crystals

Encapsulated microspheres of Pseudomonas cepacia lipase may be prepared by phase separation techniques. The crystalline LPS prepared in Example 13 may be encapsulated in polylactic-co-glycolic acid (“PLGA”) using a double emulsion method. A 700 mg aliquot of protein crystals is injected in methylene chloride containing PLGA (0.6 g PLGA/ml solvent; 10 ml). The mixture is homogenized for 30 sec at 3,000 rpm, using a homogenizer with a micro fine tip. The resulting suspension is transferred to a stirred tank (900 ml) containing 6% poly (vinyl alcohol) (“PVA”) and methylene chloride (4.5 ml). The solution is mixed at 1,000 rpm for 1 min. The microspheres in the PVA solution are precipitated by immersion in distilled water, washed and filtered. The microspheres are then washed with distilled water containing 0.1% Tween, to reduce agglomeration and dried with nitrogen for 2 days at 4° C.

### EXAMPLE 18

### Protein Content of Microspheres

The total protein content of the microspheres prepared in Example 16 may be measured using the following techniques.

Triplicate samples of 25 mg of the PLGA/PVA microspheres are incubated in 1 N sodium hydroxide with mixing for 48 hrs. The protein content is then estimated using Bradford's method (M. M. Bradford, Analytical Biochemistry, vol. 72, page 248-254 (1976)) and a commercially available kit from Byroad Laboratories (Hercules, Calif.). The protein containing microspheres are compared to PLGA microspheres without any crystals. The activity per milligram or specific activity of selected samples may also be determined.

### EXAMPLE 19

Protein Release from Microspheres

The release of protein from the PLGA microspheres prepared in Example 16 may be measured by placing 50 mg of protein encapsulated PLGA microspheres in micro centrifuge filtration tubes containing 0.22 gm filters. Next, 600 μl of release buffer (phosphate buffered saline with 0.02% Tween 20 at pH 7.4) is added to the microspheres on the retentate side of the filter. The tubes are incubated at 37° C. to allow dissolution. To measure the amount of protein released with time, samples are taken at different time intervals. The tube is centrifuged at 3000 rpm for 1 minute and the filtrate is removed for protein activity and total protein measurements. The microspheres are then resuspended with another 600 μl of release buffer.

Analysis using this example may be used to determine if the encapsulated proteins of this invention are suitable for biological delivery of therapeutic proteins. Moreover, various rates of delivery can be selected by manipulating the choice of protein crystal, size of the crystals, cross-linking of the crystals, the hydrophobic and hydrophilic characteristics of the encapsulating polymer, the number of encapsulations, dose of microspheres and other easily controllable variables.

Furthermore, the biological activity of the protein released with time may be measured using the olive oil assay for lipase microspheres, to determine if the microspheres protect and release active protein. The cumulative percent activity released, may be calculated based on the amount of input protein, and its correlation with the total protein released

### EXAMPLE 20

### Protein Release

The release of proteins from the PLGA microspheres may be measured by placing 50 mg of PLGA microspheres in micro-centrifuge filtration tubes containing 0.22 μm filters. A 600 μl aliquot of release buffer (10 mM HEPES, pH 7.4, 100 mM NaCl, 0.02% Tween, 0.02% azide) is added to suspend the microspheres on the retentate side of the filter. The tubes are sealed with 3 cc vial stoppers and covered by parafilm. The microspheres are then incubated at 37° C. Samples are taken over time by centrifugation (13,000 rpm, 1 min) of the tubes. The filtrate is removed and the microspheres are resuspended with 600 μl of the release buffer. The quality of the released protein is assayed by SEC-HPLC and enzymatic activity.

The shape and size of the protein crystals may be chosen to adjust the rate of dissolution or other properties of the protein crystal formulations of this invention.

### EXAMPLE 21

### Encapsulation of Lipase Crystals Using a Biological Polymer

Biological polymers are also useful for encapsulating protein crystals. The present example demonstrates encapsulation of cross-linked and uncross-linked crystals of Candida rugosa lipase crystals. The uncross-linked and cross-linked crystals are prepared as described in Example 1 and 15. Antibodies and chemicals were purchased from Sigma.

### A. Preparation of Coated Crystals

A solution of 1.5 ml of bovine serum albumin (“BSA”) at 10 mg/ml is prepared, in 5 mM phosphate buffer adjusted to pH 7. Next, 15 ml of a 10 mg/ml suspension of Candida rugosa lipase crystals is prepared in 5 mM K/Na phosphate buffer, 1 M NaCl, at pH 7 (“buffer”). The BSA solution is added to the crystal solution and the two solutions are mixed thoroughly. The crystals are incubated in the BSA for 30 min with slow mixing using an orbital shaker. Following the incubation with BSA, the crystals are dried overnight by vacuum filtration. The dried crystals are resuspended in buffer without albumin. The crystals are washed with buffer until no protein could be detected in the wash as measured by absorbance at 280 nm or until the A₂₈₀ nm was <0.01. The crystals are recovered by low speed centrifugation.

### B. Detection of the Albumin Coat

The coated crystals are evaluated by Western blotting to confirm the presence of the albumin layer. Following washing, coated protein crystals are incubated in 100 mM NaOH overnight to dissolve the microspheres into the constituent proteins. The samples are neutralized, filtered and analyzed by SDS-PAGE immunoblot according to Sambrook et al. “Molecular Cloning: A Laboratory Manual”, Cold Spring Harbor Laboratory Press, Cold Spring Harbor, N.Y. (1989).

The results of SDS-PAGE immunoblot of both albumin coated cross-linked and uncross-linked crystal microspheres of Candida rugosa lipase are intended to reveal a single immunoreactive species having the same molecular weight as albumin.

Samples of the albumin coated cross-linked and uncross-linked crystal microspheres of Candida rugosa lipase are then incubated with a fluorescence-labeled anti-BSA antibodies which specifically recognizes and binds to bovine serum albumin. Next, excess antibody was removed thorough washing with phosphate buffer. Microscopic examination of these fluorescently labeled albumin coated crystal microspheres under a fluorescent microscope are intended to reveal specific fluorescence-labeling of the microspheres. Uncoated lipase crystals are used as control, showing no specific binding of the antibody.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

Moreover, while we have hereinbefore described a number of embodiments of this invention, it is apparent that our basic constructions can be altered to provide other embodiments that utilize the processes and compositions of this invention.

Therefore, it will be appreciated that the scope of this invention is to be defined by the claims appended hereto and the specification as whole, rather than by the specific embodiments that have been presented hereinbefore by way of example.

We claim:

1. A nutritional product composition comprising an enzyme selected from the group consisting of a lipase, an amylase, a protease, and any combination thereof, wherein said enzyme formulated for sustained stability in an aqueous medium; and
a nutritional supplement.

2. The composition of claim 1 further comprising an aqueous medium.

3. The composition of claim 2, wherein the aqueous medium is infant formula.

4. The composition of claim 2, wherein the composition is formulated for administration to an elderly human.

5. The composition of claim 2, wherein the aqueous medium is a nutritional drink product.

6. The composition of claim 1, wherein the enzyme is present in the composition in a low-dose quantity.

7. The composition of claim 6, wherein the nutritional product is a nutrition bar.

8. The composition of claim 6, wherein the nutritional product is in powder form.

9. The composition of claims 1, wherein the composition further comprises a second lipase that is selected from the group consisting of a pre-duodenal lipase, a breast milk lipase, and a combination thereof.

10. The composition of claim 1, wherein the composition further comprises additional cofactors selected for their ability to assist in enzyme function.

11. The composition of claim 10, wherein the cofactor is a bile salt.

12. The composition of claims 1, wherein said enzyme is derived from the group consisting of bacteria cultures and mammalian cultures.

13. The composition of claim 12, wherein said enzyme is derived from Candida rugosa or functional mutants thereof.

14. The composition of claim 12, wherein said enzyme is derived from Pseudomonas cepacia or functional mutants thereof.

15. The composition of claim 12, wherein the enzyme is a pancreatic enzyme.

16. The composition of claim 1, wherein the composition comprises components selected from the group consisting of water, enzymatically Hydrolyzed Reduced Minerals Whey Protein Concentrate, Vegetable Oils, Lactose, Corn Maltodextrin, and less than 1.5% of: Potassium Citrate, Potassium Phosphate, Calcium Chloride, Calcium Phosphate, Sodium Citrate, Magnesium Chloride, Ferrous Sulfate, Zinc Sulfate, Sodium Chloride, Copper Sulfate, Potassium Iodide, Manganese Sulfate, Vitamins, Taurine, Nucleotides, L-Carnitine, and combinations thereof.

17. An infant formula composition comprising infant formula and an enzyme selected from the group consisting of a lipase, an amylase, a protease, and any combination thereof, said enzyme formulated for sustained stability in the infant formula.

18. A packaged infant formula additive comprising an enzyme selected from the group consisting of a lipase, an amylase, a protease, and any combination thereof, formulated for sustained stability in infant formula; and
instructions for mixing the additive with infant formula and administration of the resulting product mixture to an infant.

19. A composition useful for increased intestinal absorption of a nutrient comprising a low-dose quantity of an enzyme selected from the group consisting of a lipase, an amylase, a protease, and any combination thereof formulated for low-dose administration of the enzyme to a subject in aqueous medium.

20. A digestion enhancement composition comprising a low-dose quantity of an enzyme selected from the group consisting of a lipase, an amylase, a protease, and any combination thereof
formulated for low-dose administration of the enzyme to a subject in aqueous medium.

21. A method of increasing intestinal absorption of a nutrient comprising administering to an infant an enzyme selected from the group consisting of a lipase, an amylase, a protease, and any combination thereof,
the enzyme formulated for sustained stability in an aqueous medium, and adapted for administration to an infant in said aqueous medium (e.g., infant formula),
such that the intestinal absorption of the nutrient in the infant is increased.

22. The method of claim 21, wherein increasing intestinal absorption results from increased catabolism of fats and proteins in the gastrointestinal pathway.

23. The method of claim 21, wherein the increase in intestinal absorption is measured by an increase in height and/or weight.

24. The method of claim 21, wherein the increase in intestinal absorption is measured by a decrease in a symptom of pancreatic insufficiency.

25. The method of claim 24, wherein the symptom of pancreatic insufficiency are selected from the group consisting of bloating, colic, and diarrhea.
